(19) United States
(12) Patent Application Publication
(10) Pub. No.: US 2004/0185513 A1
(43) Pub. Date: Sep. 23, 2004

(54) Method for enhanced generation of biomarkers for mass spectrometry detection and identificaiton of microorganisms

(76) Inventor: Plamen A. Demirev, Ellicott City, MD (US)

Correspondence Address: Francis A. Cooch, Office of Patent Counsel, The Johns Hopkins University, Applied Physics Laboratory, 11100 Johns Hopkins Road, Laurel, MD, 20723-6099, US

(21) Appl. No.: 10/767,910

(22) Filed: Jan. 29, 2004

Related U.S. Application Data
(60) Provisional application No. 60/446,820, filed on Feb. 12, 2003.
(60) Provisional application No. 60/487,414, filed on Jul. 15, 2003.

(51) Int. Cl. G01N 33/53; G01N 33/569; G01N 33/554

(52) U.S. Cl. 435/7.31; 435/7.32

(57) ABSTRACT

A method for identifying small acid-soluble proteins (SASPs) by generating an increased number of biomarkers upon controllably triggering enzymatic digestion in an intact spore is disclosed. An additional embodiment of the method includes oxidizing an unknown protein in a microorganism by pre-selected oxidation facilitating agent, which causes a predetermined mass gain in Methionine, thus serving as an indicator of a particular family of proteins.

### CROSS-REFERENCE TO RELATED APPLICATIONS

This application claims the benefit of U.S. Provisional Application No. 60/446,820, filed Feb. 12, 2003, and U.S. Provisional Application No. 60/487,414, filed Jul. 15, 2003, the contents of each being incorporated herein by reference.

### BACKGROUND OF THE INVENTION

1. Field of the Invention

The present invention generally relates to methods for affecting unknown microorganisms in a manner leading to generation of the increased number of biomarkers, further used for rapid and reliable identification of these microorganisms.

2. Description of the Related Art

Rapid and accurate microbial identification is critical in diagnosing diseases, predicting on-coming public health hazards, monitoring potential food contamination, regulating bioprocessing operations and recognizing bio-warfare threats. Typically, cells constituting each microorganism are vegetative cells that are actively growing. However, placed under unfavorable conditions, these cells undergo sporulation, a process during which a vegetative cell produces a spore in an asymmetric cell division. The spore is an environmentally-resistant dormant and reproductive body produced by certain Gram-positive microorganisms. Left alone, the spores do not pose a threat; yet, they will “spring back to life” turning into vegetative cells if the conditions are favorable. This process is called germination. Hence, detection of both the vegetative cells and/or the spores is important. Spores, unlike the cells, do not break into molecules easily, thus making the microorganism identification process rather difficult.

The classes of molecules present in the spores include lipids (e.g., phospholipids), proteins, nucleic acids (DNA and RNA), and small molecules (e.g., dipicolinic acid). Proteins, currently being exploited, contribute up to 50% of the dry weight distributed among 200-6000 molecular species in bacteria. The nucleic acids contribute only up to about 0.01% of the dry weigh. DNA constitutes the most unique characteristic for each microorganism; however, there is only one copy per cell without amplification. Thus, with current instrumentation, proteins provide the most characteristic biomarkers accessible in the analysis of intact organisms by mass spectrometry.

Several instrumental analytical techniques including field, plasma and laser desorption, secondary ion mass spectrometry (SIMS), and fast atom bombardment were developed in the past to enhance the speed and accuracy of identification of bacterial cells. Generally, each of these techniques is associated with mass spectrometry (MS), which is based on determining chemotaxonomic markers, or biomarkers, specific for each bacteria species. The biomarkers may be any one or a combination of the classes of molecules present in the spore. MS provides identification of chemical structures, the determination of the compositions of mixtures, and qualitative and/or quantitative elemental analysis. In operation, a mass spectrometer generates ions of sample molecules under investigation, separates the ions according to their mass-to-charge ratio (m/z), and measures the relative abundance of each type of ions. This analysis of the mass distribution of the molecule and its ion fragments can lead to a molecular “fingerprint”, (biomarker signature), for identification of a given microorganism.

Matrix-assisted laser desorption/ionization (MALDI) has become one of the most important ionization methods used for biological mass spectrometry in conjunction with a time-of-flight (TOF) mass spectrometer. The requirements for the MALDI technique include absorption of the laser light by the analyte-matrix mixture, promotion of ionization and dispersion of the energy deposited in the sample in order to produce intact molecular ions from the analyte. Mass spectrometers, performing MALDI are commercially available and can be equipped, for example, with single or multiple quadrupole, single or multiple magnetic sector, Fourier Transform ion cyclotron resonance (FTICR), ion trap, and combinations thereof (e.g., ion-trap/TOF) mass analyzers. Overall, this method provides useful structural information and is used for directly obtaining mass spectra from polar, high molecular weight compounds encountered in microbiological studies without resorting to more destructive techniques. MALDI has been used to produce molecular biomarker ions from intact microorganisms, wherein “intact” refers to microbial cells suspended in a solution and/or deposited directly on the sample holder.

None of the mass spectrometry techniques is particularly useful if applied for investigating bacterial spores due to the difficulty associated with the release of cortex/core proteins to be detected as biomarkers in a mass spectrum. One of the reasons is that spores of Bacillus, for example, are both biologically dormant and much more resistant than vegetating cells to a variety of environmental stress factors. Among known strategies directed to identification of Bacillus spores, one relies on the extraction of small, acid-soluble spore peptides (SASPs) by utilizing acid treatment. Further, an external proteolytic enzyme, such as bovine trypsin, is added to digest the proteins before the mass spectrometry is performed. [See, e.g., Characterization of Bacillus Spore Species and Their Mixtures Using Postsource Decay with a Curved-Field Reflection, Warscheid, B; Fenselau, C. Analytical Chemistry]

However, this approach is associated with certain inconveniences. Firstly, external enzymes require a special storage (e.g., refrigeration), and it may not be an easy task to accomplish. Secondly, the addition of an external element is time consuming, it requires special conditions (temperature, humidity, pH). This could be a huge detriment in a field situation, typically requiring rapid identification of potentially dangerous microorganisms. Thirdly, generation of non-biomarkers tryptic fragments from autolysis may reduce specificity during identification of SASPs, thus jeopardizing the reliability of the performed test.

It is known that some proteins undergo certain spectral modifications in response to chemical regents or other external agents. See, e.g., Setlow et al., Germination of spores of Bacillus subtilis with dodecylamine, Journal of Applied Microbiology 95: pp. 637-648, (2003). In the context of mass spectrometry, these changes may be manifested by the presence of closely positioned peaks or doublets, attributed to certain amino acids. However, to the best of the applicant's knowledge, this phenomenon has not been implemented till now for facilitating the identification of microorganisms by MS. Yet, a high probability of the presence of the specific amino acid in a protein from otherwise unknown microorganisms may exclude a great number of proteins not having this amino acid during the identification search, which is, thus, can be much more efficient.

It would be desirable to provide a simple and efficient method for increasing the number of biomarkers in response to controllably affecting unknown microorganisms and using these biomarkers for rapid identification of these microorganisms.

### SUMMARY OF THE INVENTION

This feature is attained by the methods of the present invention designed, in one of its embodiments, to create conditions capable of triggering the germination process of unknown spores associated with the increased number of biomarkers, which are detected during mass spectrometry. In accordance with another embodiment of the present invention, the above-formulated feature is attained by oxidizing a particular amino acid in the proteins of an unknown microorganism, which releases proteins known to undergo certain chemical changes that are manifested by the increased number of biomarkers. In either of these embodiments, the increased number of biomarkers serves to facilitate a microorganism's identification.

Controllable inducement or triggering the germination of spores in the first embodiment of the invention is accompanied by activation of a certain family of proteins causing internal enzymatic digestion and release of tryptic peptides, which are detected by mass spectrometry. The mechanism of this inventive aspect is based on the fact that the cortex/core of a spore contains such necessary enzymes, which, if the spore is triggered into germination, are capable of digesting proteins much like external enzymes. Peptides, derived from small acid-soluble proteins (SASPs), derived in great numbers as a result of the internal enzymatic digestion, are proved to be reliable biomarkers leading to rapid and efficient identification of microorganisms.

In accordance with the other embodiment of the present invention, two samples of untreated and chemically-treated unknown microorganism are analyzed by mass spectrometry and, based on the presence of doublets, a probability is assigned as to the presence and the number of the specific amino acid, Methionine, in the proteins of the tested microorganism. This probability is further used for excluding numerous proteins that do not have this concrete amino acid during the microorganism identification search.

In particular, this aspect of the invention includes a method of aiding in the identification of identification of a microorganism by oxidizing one of two samples of the same microorganism and further obtaining mass spectra for each of the samples. If a predetermined mass shift between a respective pair of related biomarkers appearing on the mass spectra is observed, then it is attributed to the presence of a known amino acid in a respective protein. This valuable piece of information is used in searching for a group or family of proteins, including the known amino acid or having the same relative number of known amino acid residues. The search is performed by excluding therefrom all proteins, which do not contain the known amino acid, or the relative same number of known amino acid residues, thereby eliminating proteins which are unrelated or which do not correspond to the respective protein.

The principle feature of the present invention is to provide reliable, time-efficient and simple methods for increasing biomarkers used for rapid identification of microorganisms.

Another feature of the present invention is to provide a method of triggering conditions favorable to the germination of spores in an efficient, simple and controlled manner.

A further feature of the present invention is to provide a method of detecting SASPs peptides released by the spores during the induced germination process by utilizing mass spectrometry.

Still another feature of the present invention is to provide a method of identifying spores based on detected masses of SASPs and their fragments.

Yet a further feature of the invention is to provide a method for inducing a chemical reaction in unknown microorganisms accompanied by the generation of the increased number of biomarkers, which are further used for a rapid identification search of these microorganisms.

### BRIEF DESCRIPTION OF THE DRAWINGS

The above and other objects, features and advantages will become more readily apparent from the following description, references being made to the accompanying drawings, in which:

FIG. 1 is a view of Bacillus cereus T spore;

FIG. 2 is a flow chart illustrating an embodiment of the methods of the present invention;

FIG. 2A illustrates a partial sequence (consensus motif) of SASP cleaved by internal germination protease (GRP);

FIG. 3 shows the positive MALDI-TOF spectra from intact Bacillus-globigii spores obtained as a function of different “trigger” times, during which the collected spores were exposed to the germination process;

FIG. 4 illustrates the negative MALDI-TOF spectra from intact Bacillus-globigii spores obtained as a function of different “trigger” times, during which the collected spores were exposed to the germination process

FIG. 5 shows the positive MALDI-TOF spectra from intact Bacillus-subtilis spores obtained as a function of different “trigger” times, during which the collected spores were exposed to the germination process;

FIG. 6 illustrates the negative MALDI-TOF spectra from intact Bacillus-subtilis spores obtained as a function of different “trigger” times, during which the collected spores were exposed to the germination process;

FIGS. 7A and 7B illustrate the MALDI-TOF spectra of the Bacillus-cereus T spores obtained under control and upon addition of an oxidizing agent (TFA), respectively;

FIG. 8 is a part of SwissProt/TrEMBL database identifying proteins based on the obtained molecule weight of biomarkers shown in FIG. 7A;

FIG. 9 illustrates amino acid sequences of the major SASP biomarkers in the MALDI spectrum of intact Bacillus-cereus T spores; and,

FIG. 10 illustrates the MALDI-TOF mass spectra of the Bacillus-cereus T spores treated with a relatively strong oxidizing agent (H₂O₂).

### DETAILED DESCRIPTION OF THE PREFERRED EMBODIMENTS

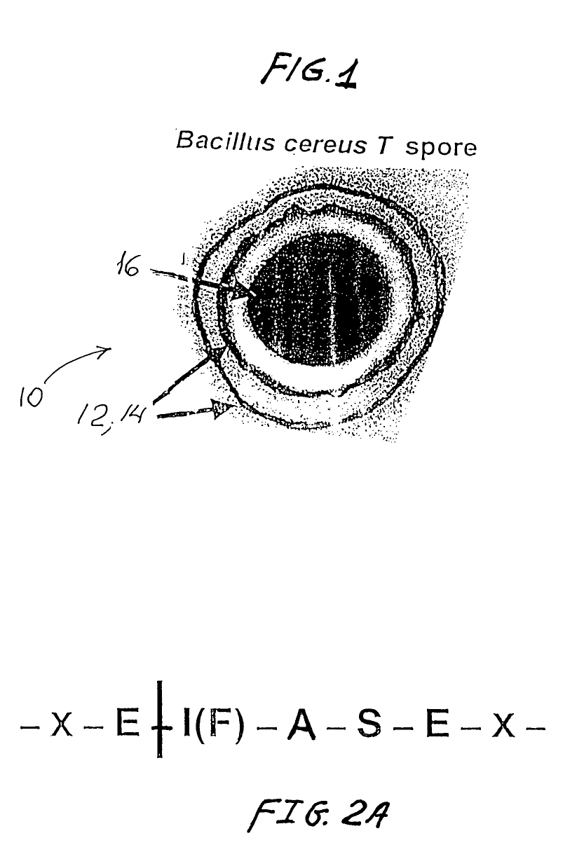

Referring to FIG. 1, a spore 10, such as a Bacillus cereus T spore, has a plurality of concentric coats 12, 14 and a core or cortex 16 containing a genetic make-up of the spore 10. Among other classes of molecules constituting the cortex 16 of the spore, proteins are large, complex molecules that carry out the tasks of life. Each protein is initially formed as a string of amino acids whose identity and order are dictated by a gene according to the sequence of its DNA bases.

Each type of Bacillus spore produces unique SASP proteins allowing identification of the microorganism. One of the most critical applications of the correct identification of microorganisms, such as Bacillus cereus spores, Bacillus anthracis spores and the like, allows for an early warning about the proximity of biological warfare weapons.

In accordance with one aspect of the method of the present invention, it has been found that the germination state of the spore, during which the spore breaks its dormancy state and rapidly looses its resistance properties, is accompanied by the digestion of a family of proteins, referred to as small, acid-soluble spore peptides (SASPs), from the cortex/core without the use of the external enzymes, e.g., trypsin, as known in the art. SASP has a variety of functions including, but not limited to, binding to double-stranded spore DNA to change it to A-form, protecting DNA from chemical and enzymatic cleavage and UV light, and degrading during an initial period of germination to provide amino acids for both de novo protein synthesis and for other metabolism processes in the germinating spore.

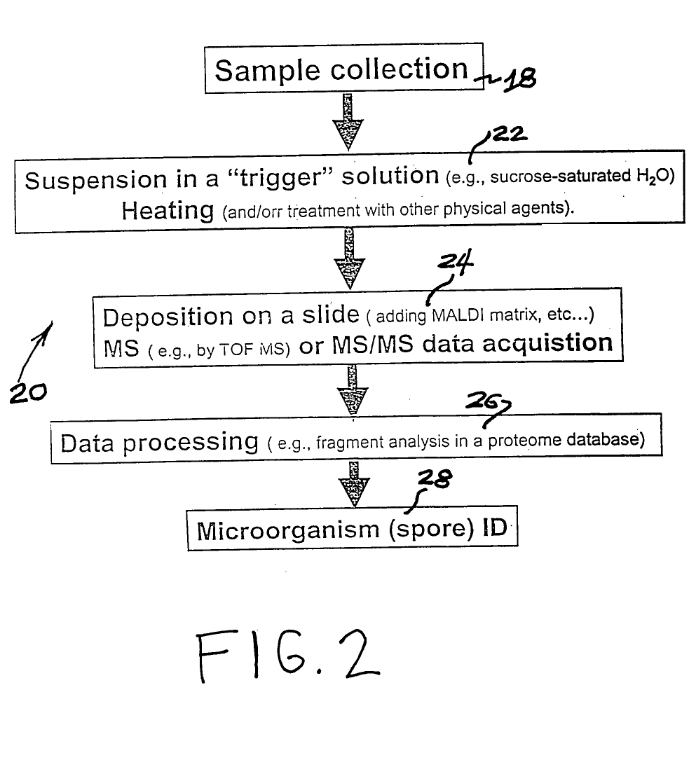

Turning to FIG. 2, the inventive method 20 is based on the fact that the spore 10 includes internal enzymes, such as germination protease (GPR), which, once the germination process of the spore begins, will digest (“chop” down) SASPs similarly to the external enzymes without fractionation or isolation. In particular, the GRP acts on SASPs via a specific cleavage in accordance with the general consensus sequence motif, as shown in FIG. 2A. As a result, the partial sequencing of proteolytic. peptides derived from the abundant SASP proteins, can be further used for rapid identification of spores by a variety of mass spectrometry and statistical methods. In accordance with the inventive concept, the germination state of the spore is artificially induced by a variety of triggering factors and is characterized by the release of SASPs proteolytic peptides during digestion of the larger intact proteins by the internal enzyme GRP.

Sequentially, the method of the present invention begins with a sample collection step 18, which may be performed in a variety of ways. For example, spores may be grown on Agar plates or Petri plates containing sporulation media, further harvested and, finally, may be stored at the sub-zero temperatures. Aerosolized spores can be collected and concentrated from air, as known in the art.

As further shown in FIG. 2, a triggering step 22 is directed to breaking the dormancy of the collected spores, which loose their resistance properties in the process of germination. A number of physical, chemical and combined physical and chemical methods can trigger the germination process. Typically, the germination process encompasses a sequence of phases beginning with physical modifications of the spore, such as formation of small cracks in the spore coats 14, 16 and enlargement. Physical trigger processes including, but not limited to, temperature and pressure increase, sonification, and the like are certainly able to stimulate this phase of the germination.

The germination phase following the initial physical modifications is particularly significant in the context of this invention because the internal enzymes including the GPR are induced to digest the SASPs into smaller peptide sequences. Among the chemicals capable of enhancing the triggering of the germination process, the amino acid Ala, “AGFK”—a combination of asparagin (Asn), glutamic acid (Glu), Fru, and potassium (K⁺), nutrients, including other amino acids, purine nucleosides and sugars and other cations, have been proven to be effective, as disclosed in the above referenced article by Setlow et al. Critically, all of these and other spore germination triggers interact with the GRP, which starts digesting the cortex's large pool of SASPs by breaking the latter in accordance with the sequence of FIG. 2A.

Based on the above-discussed methodology, triggering step 22 of FIG. 2 includes at least suspending the collected spores in a trigger solution such as, for example, sucrose-saturated water, at elevated temperatures for a sufficient period of time ranging from, for example, about several seconds to about a quarter of an hour (about 15 minutes). Advantageously, the spores are suspended in the triggering solution in a ratio of about 2:1 and further heated up to a suitable temperature, e.g., from about 40 to about 60° C. for a sufficient time period, e.g., about 5 to about 15 minutes. As one skilled in the art will readily appreciate, all physical parameters including the time intervals and temperature ranges are subject to changes depending on the given conditions.

While the germination process may last for hours, its initial digestive phase ordinarily occurs during the first minutes and is sufficient to provide the necessary information for further identification of released SASPs peptides. As discussed above, this phase is characterized by the degradation of the spore's large amount of SASPs peptides released through the cracks in the spore coats. The small size of cleaved SASPs peptides leads to a greater number of detected biomarkers during a mass spectrometry step 26 (FIG. 2), which, in turn, enhances the identification of spores performed during step 28.

Returning to the step 22 of FIG. 2, a trigger solution can be prepared and heated with the collected spores in a separate bath and is further added to MALDI matrix on a slide of MALDI-TOF, as illustrated in step 24. However, it is possible to prepare the triggering suspension directly on the sample slide.

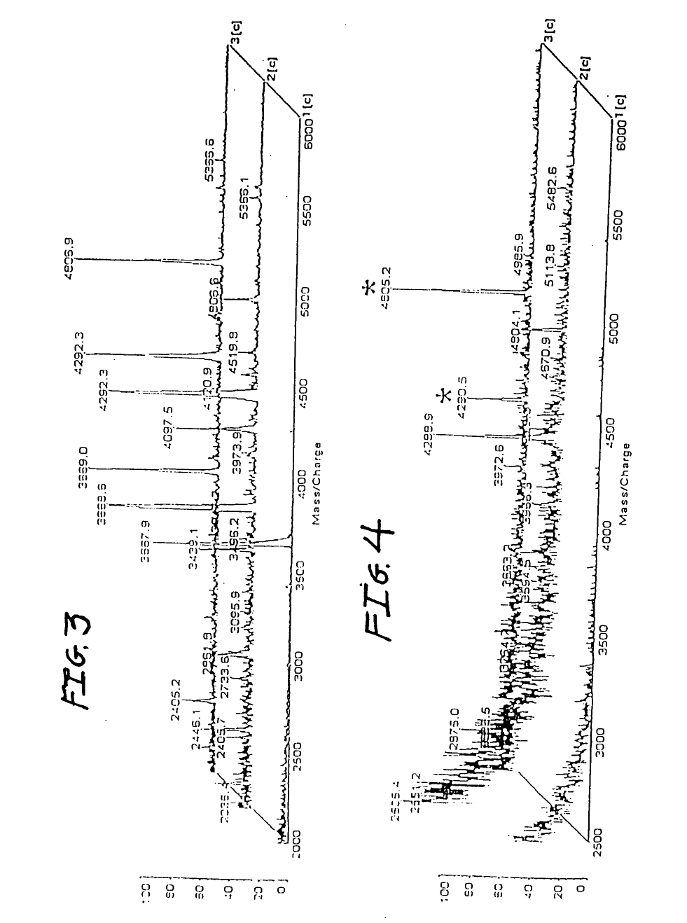

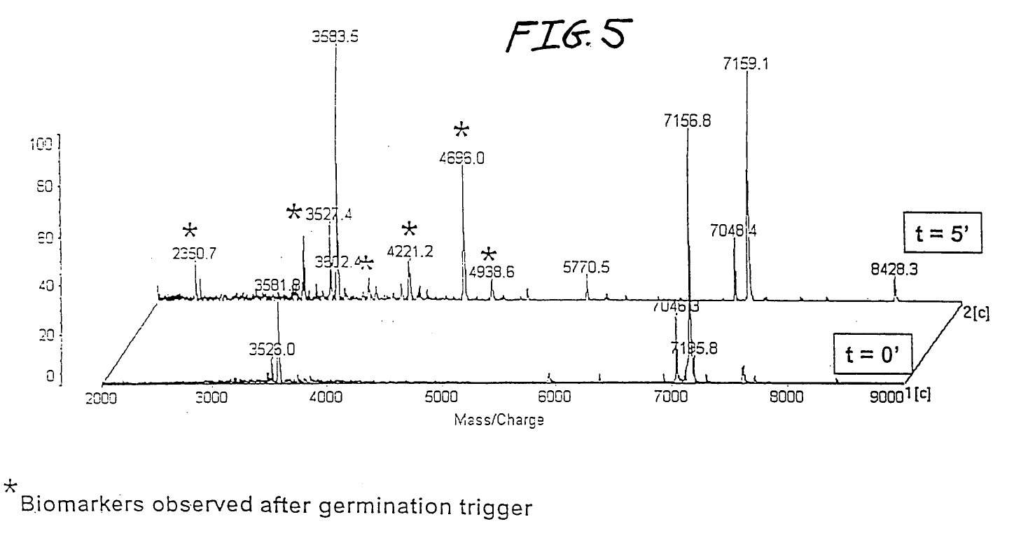

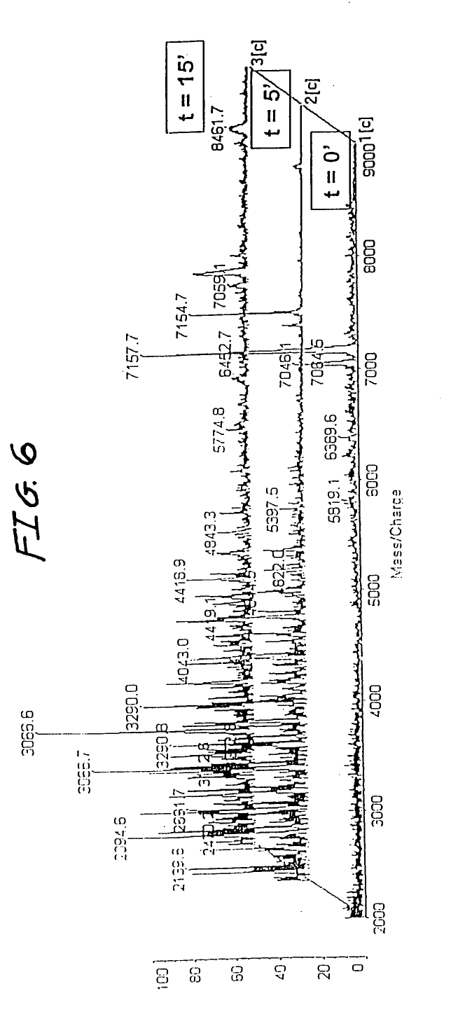

Turning now to FIGS. 3-6, it can be seen that the release of SASPs during the germination process is accompanied by numerous additional biomarkers. Thus, as shown in FIG. 3, B.globigii biomarker profile has been obtained for the positively charged ions desorbed from the B.globigii spore exposed to the triggering solution at elevated temperatures for increasingly longer time periods since the beginning of the germination process. The number of detected biomarkers steadily increases as the “trigger” time of the germination process increases. The same conclusion can be made based on the B.globigii biomarker profile obtained for the negatively charged ions, as shown in FIG. 4. Similar results illustrating a great number of biomarkers have been obtained during the germination process of B.subtilis spores, as shown in FIGS. 5 and 6, illustrating the mass spectra obtained for the positively charged ions and negatively charged ions, respectively. Treating the spores for longer trigger “time” periods may lead to a gradual decrease of the detected biomarkers, which eventually-will not be detected at all. There is a high probability that the inability to detect biomarkers from SASPs after triggering of germination may be a reliable indicator of the dead spore.

It is to be understood that the invention is not limited exclusively to the MALDI-TOF mass techniques which, of course, may be practiced in combination with other ionization methods, such as, for example, fast atom bombardment, plasma desorption, electrospray ionization, or massive cluster impact ionization, and mass analyzers, including tandem mass spectrometry.

Identification of the spores can be performed by various statistical methods. One of the methods may include identification of the molecular weight of each detected fragment and sum up the weights of all fragments to identify a given protein. However, it is not unusual to have several different proteins basically having the same molecular weight within experimental mass accuracy, but a completely different sequence of fragments. Thus, a simple arithmetic addition of fragmental molecular weights may not be a reliable identification of the whole protein. Recently, a new bioinformatics-based approach has been put forward, which characterizes microorganisms based on matching protein molecular masses in the spectrum with protein molecular masses predicted from already sequenced genomes, as disclosed by C. Fenselau and P. A. Demirev, “Characterization of intact microorganisms by MALDI MS.” (2002 John Wiley & Sons, In., Mass Spectrometry Reviews 20:175-171, 2001). This method is, however, limited only to those microorganisms whose genomes are sequenced. The flexibility of this approach allows interpretation of the biomarker spectrum rather than matching or correlating it. Since fragmentation of the large protein molecules by known enzymes is sequence specific, comparing identified and calculated peptide fragments, if, of course, the compared masses match. Knowing this sequence can eventually lead to a reliable identification of microorganisms.

In summary, the method of the present invention is directed to triggering of external digestion of different types of unknown spores provides for more and different biomarkers making further identification of SASPs and microorganisms more reliable. Furthermore, since the inventive method does not require external enzymes, it avoids logistic problems associated with storing the external enzyme(s) and adding it to a suspension of spores to be examined. Also, the use of the method of the present invention combined with bioinformatics-aided identification of proteins may verify the correctness of SASP sequences in SwissProt and other known protein databases. Overall, the internal digestion of SASPs in spores conducted in accordance with the inventive method facilitates their correct identification leading to the identification of microorganisms.

A further aspect of the invention is concerned with a procedure providing a time efficient process for identifying microorganisms based on mass spectrometry. Similarly to the previously disclosed inventive aspect, this embodiment of the invention is based on obtaining a greater number of biomarkers due to a chemical reaction of unknown proteins with certain oxidizers, causing these proteins, which may contain Methionine (Met), to increase their molecular mass with a predetermined value. This mass increase—shift in biomarker mass between treated and untreated samples, indicates the number of Met residues. As a consequence, one may assume that the protein to be identified indeed has the presumed number of Met residues and search only for a family of proteins containing Met in general and, further, for a family of proteins with the projected number of Met residues, while excluding those proteins that do not have this amino acid or the projected number of amino acid residues thereof from his/her search.

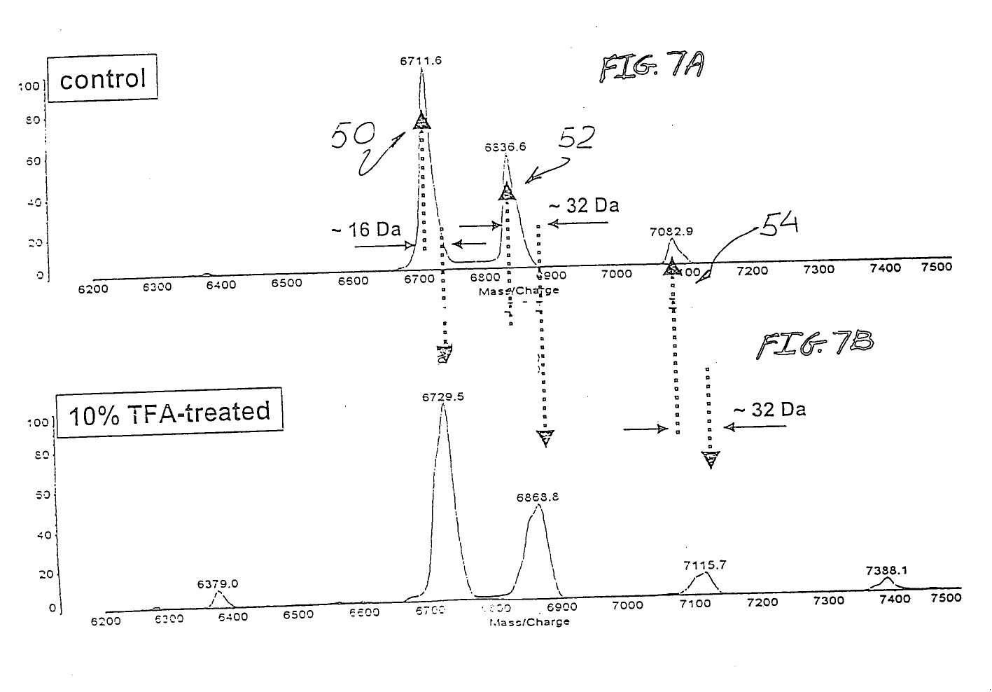

In accordance with the method of the present invention, upon preparing a suspension of spores under the controlled conditions such as, for example, about. 0.3 μl B. cereus T spore suspension mixed with an about 0.3 μl α-CHCA matrix, a part thereof can be further treated by an oxidation-facilitating agent to facilitate Met oxidation. Utilizing the MALDI-TOF technique, the control and oxidized samples are tested in parallel, and two mass spectra, as shown in FIG. 7A and 7B, are obtained. The spectrum of the untreated suspension under control is illustrated in FIG. 7A and is characterized by three biomarkers 50, 52, and 54. Note that the biomarker 50 has a peak corresponding to about 6711.6, the following biomarker 52 has a mass approximating 6,835.6 and the biomarker 54 corresponds to 7082.9. The mass spectra of the prepared sample treated with an oxidation-facilitating agent, e.g., trifluoroacetic acid (TFA), is illustrated in FIG. 7B. As can be seen from the latter, the biomarkers 50, 52, and 54 have been shifted by 16 Da, 32 Da and 32 Da, respectively. The mass shift is not arbitrary; it is known that Met gains a certain molecular weight during the oxidation reaction. As a result, one can assume with a relatively high degree of probability that the protein corresponding to the biomarker contains one Met, while the second and third biomarkers 52 and 54 each have two Met residues, for the reasons explained below.

Based on the assumption regarding the number of Met residues and armed with the observed masses of the detected biomarkers, one can now search different protein databases or protein fingerprint libraries using this information to exclude a great number of the proteins that do not have the approximate same number of Met residues. As a consequence, the field of search is narrowed and, thus, much more time efficient.

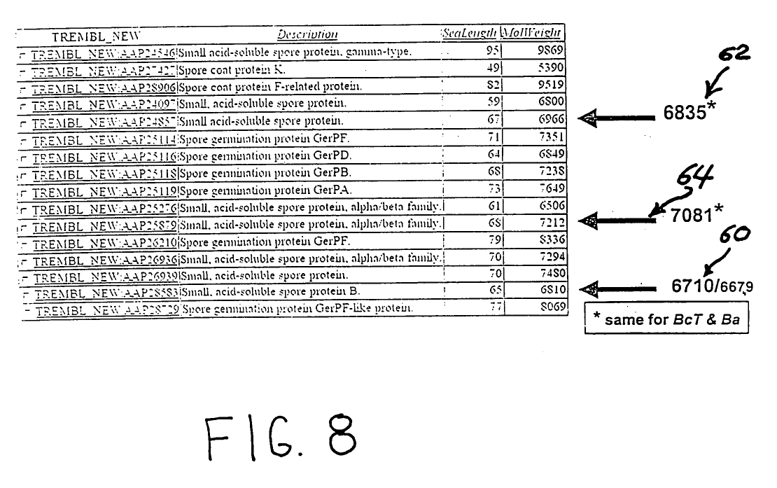

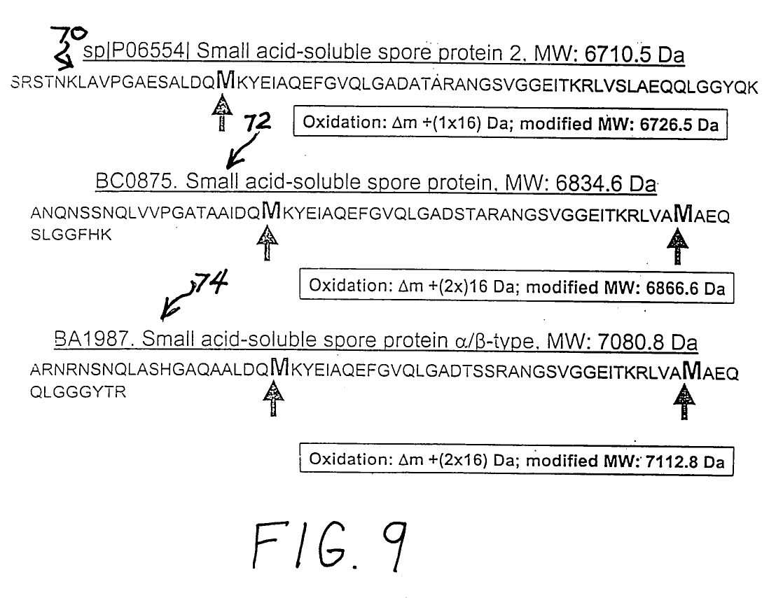

The use of protein database for identification of the proteins corresponding to the identified biomarkers 50, 52 and 54 is illustrated in FIG. 8 illustrating a relevant part of the database, which indicates that each of the observed biomarkers is described as Small acid-soluble proteins (SASP) 60, 62 and 64. Since these proteins have been already sequenced at 70, 72 and 74, as shown in FIG. 9, the assumption, made during the initial evaluation of the mass spectra in FIGS. 7A and 7B, is fully reinforced. In particular, the sequence 70 corresponding to the detected biomarker 50 indeed has one Met residue, while the sequences 72 and 74 of the biomarkers 52 and 54 (FIG. 7A), respectively, each have two Met residues.

An oxidation facilitating agent advantageously assists in determining the mass shift. The oxidation of Met by addition of, for example, TFA, results in a MetO (sulfoxide) compound known to increase the molecular weigh of Met by 16 Da. As one skilled in the art will readily appreciate, other oxidation facilitating agents known to one skilled in the art can be used herein. Naturally, if an unknown protein is represented by a 32 Da mass shift, as is the case with biomarkers 52 and 54, knowing that the agent is TFA, it is highly probable that the protein has two Met residues summed up to give the 32 Da mass shift. Thus, any mass shift (αM) due to the TFA oxidation of an unknown protein that can be calculated by the following formula

ΔM˜16×N

wherein N is an integer of the projected number of Met residues, and represents a highly probable basis for excluding proteins not having Met from the identification search.

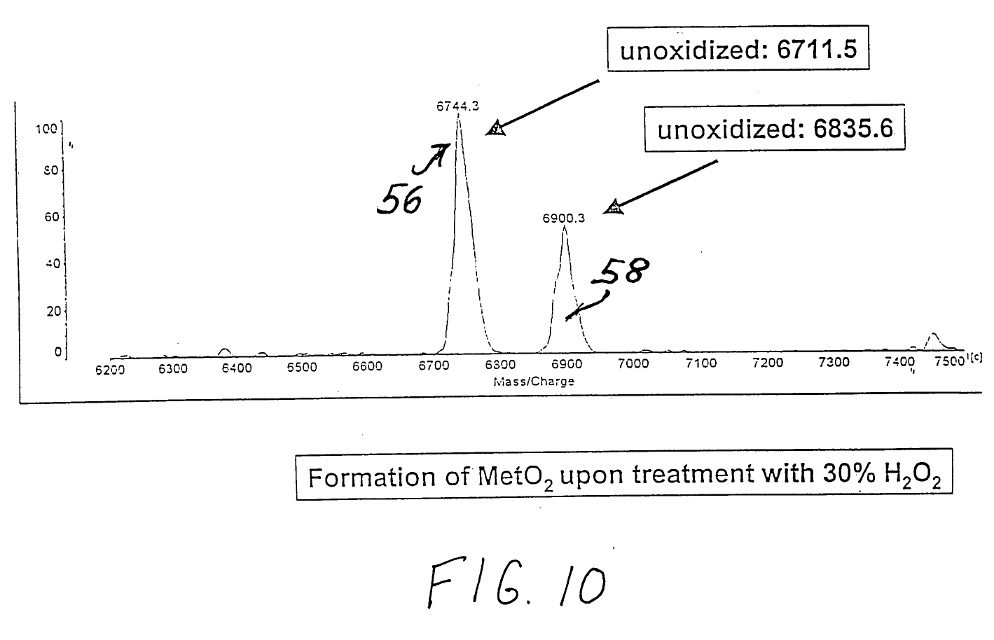

However, as shown in FIG. 10, instead of a relatively weak TFA, a stronger oxidation-facilitating agent, e.g., about 30% v hydrogen peroxide (H₂O₂), may be used to react chemically with Met turning the latter into MetO₂ (sulfone). In this case, as is known, every Met residue affected by the oxidation would gain approximately 32 Da of molecular weight. Thus, a formula used in conjunction with H₂0₂ will be as follows:

≢M˜32×N

wherein N has the aforementioned meaning.

As a result, the protein represented by a biomarker 56 would have only one Met residue, because the mass spectrum shows a 32 Da mass shift between the treated and untreated samples, whereas the protein corresponding to the biomarker 58 is presumed to have three Met residues, since the mass shift approaches 76 Da.

If the proteins are not sequenced, the search based on the bioinformatics databases, including, but not limited to, SwissProt and TrEMBL, Gene Bank, DNA Data Bank of Japan and others, is fruitless. As an alternative manner of identifying microorganisms based on the presumed number of Met due to the oxidation reaction, the identification step can be done by using a variety of reference protein fingerprint libraries containing a large number of mass spectra (signatures) of microorganisms. The algorithms used to find the best match between the observed and a library spectrum are known and discussed in the above-referenced article by C. Fenselau and P. A. Demirev. Of course, for successful identification by the library matching approach, it would be necessary to oxidize the known microorganisms in order to deposit both spectra and to verify the match.

While the above description contains many specifics, these specifics should not be construed, as limitations of the invention, but merely as exemplifications of preferred embodiments thereof. Those skilled in the art will envision many other embodiments within the scope and spirit of the invention as defined by the claims appended hereto.

What is claimed is:

1. A method for generating biomarkers from spores, comprising the steps of:
(a) triggering spore germination, thereby inducing digestion of small acid-soluble proteins (SASPs); and, (b) detecting biomarkers generated by the SASPs digestion and released during the step (a) by utilizing mass spectrometry, thereby obtaining mass spectra of the released peptides from SASPs.

2. The method of claim 1, wherein step (a) includes a step of chemically treating the spores with a germination-triggering agent.

3. The method of claim 2, wherein the spores are suspended in a trigger solution in a ratio of about 2:1.

4. The method of claim 3, wherein the triggering agent induces an internal enzyme located in a core of the suspended spores to digest the SASPs, breaking them into peptide fragments with predetermined amino acid sequences.

5. The method of claim 4, wherein the triggering agent is selected from the group consisting of Ala, a combination of Asn, Glu, Fru, K⁺, purine nucleosides, sugars, cations and combinations thereof.

6. The method of claim 3, wherein the trigger solution is a saturated water solution of sucrose.

7. The method of claim 4, wherein the internal enzyme is germination protease (GRP).

8. The method of claim 2, wherein step (a) includes a step of physically treating the spores before chemical treatment.

9. The method of claim 8, wherein the step of physically treating the spore is selected from the group consisting of sonification, elevated pressures, elevated temperatures and combinations thereof.

10. The method of claim 2, wherein the spores are suspended in the trigger solution and heated to about 60° C. for a predetermined period of time.

11. The method of claim 10, wherein the predetermined period of time lasts between about a few seconds and about one hour.

12. The method of claim 1, wherein step (b) includes matrix-assisted laser desorption/ionization mass spectrometry (MALDI MS) or tandem mass spectrometry (MS/MS).

13. The method of claim 4, further comprising the step of identifying the fragments of the digested SASPs by bioinformatics.

14. The method of claim 13, further comprising the step of identifying the spores.

15. A method of aiding in the identification of a microorganism comprising the steps of:
(a) preparing at least two samples of the microorganism;
(b) oxidizing one of the at least two samples of the microorganisms;
(c) obtaining mass spectra for each of the at least two samples, thereby generating a first and second plurality of biomarkers produced by the non-oxidized and oxidized samples of the microorganism, respectively;
(d) observing a predetermined mass shift between a respective pair of the biomarkers of the first and second plurality of biomarkers, wherein the predetermined mass shift is attributed to the presence of a known amino acid in a respective protein; and,
(e) searching for a group or family of proteins, wherein the group or family of proteins includes the known amino acid or wherein the group or family of proteins has the same relative number of known amino acid residues, wherein the searching is performed by excluding from the search all proteins which do not contain the known amino acid, or the relative same number of known amino acid residues, thereby eliminating proteins which are unrelated or which do not correspond to the respective protein.

16. The method of claim 15, wherein the known amino acid is Methionine (Met).

17. The method of claim 16, wherein the step of oxidizing includes adding trifluoroacetic acid (TFA) to the one of the at least two samples of the microorganism, wherein the predetermined mass shift corresponds to about 16 Da for each Met residue present in the sample.

18. The method of claim 16, wherein the step of oxidizing includes adding hydrogen peroxide (H₂O₂) to the one of the at least two samples of the microorganism, wherein the predetermined mass shift corresponds to about 32 Da for each Met.

19. The method of claim 15, wherein the step of searching for the group or family of proteins includes screening a protein sequence database or a library of protein fingerprints.

20. The method of claim 15, wherein the mass spectra are obtained by MALDI-TOF mass spectrometer.
